(19) United States
(12) Patent Application Publication
(10) Pub. No.: US 2014/0079665 A1
(43) Pub. Date: Mar. 20, 2014

(54) THERAPEUTIC ANTI-IGF1R COMBINATIONS

(71) Applicant: MERCK SHARP & DOHME CORP., Rahway, NJ (US)

(72) Inventors: Liliane Goetsch, Ayze (FR); Karl Hsu, Cambridge, MA (US)

(21) Appl. No.: 14/118,007

(22) PCT Filed: May 15, 2012
(86) PCT No.: PCT/US2012/037864

§ 371 Date: Nov. 15, 2013

Related U.S. Application Data
(60) Provisional application No. 61/487,452, filed on May 18, 2011.

(51) Int. Cl. C07K 16/32; A61K 45/06; A61K 31/4745; A61K 31/7068; A61K 39/395

(52) U.S. Cl. CPC C07K 16/32; A61K 31/7068; A61K 39/39558; A61K 31/4745; A61K 45/06
USPC 424/85.2; 424/133.1; 424/130.1; 424/85.4

(57) ABSTRACT

The present invention provides, in part, compositions including an anti-IGF1R antibody or antigen-binding fragment thereof and any one or more of cisplatin, pemetrexed, gemcitabine and/or irinotecan; optionally including or excluding a further chemotherapeutic agent, as well as methods of treating diseases, such as cancer, using such compositions.

This application claims the benefit of U.S. Provisional Patent Application No. 61/487,452; filed May 18, 2011; which is herein incorporated by reference in its entirety.

### FIELD OF THE INVENTION

The field of the invention relates to methods for treating diseases such as cancer with combinations that include an anti-IGF1R antibody and cisplatin, pemetrexed, gemcitabine and/or irinotecan.

### BACKGROUND OF THE INVENTION

The insulin-like growth factors include insulin-like growth factor-I (IGF-I) and insulin-like growth factor-II (IGF-II). These growth factors exert mitogenic activity on various cell types, including tumor cells, by binding to a common receptor named the insulin-like growth factor receptor-1 (IGFR1).

Several lines of evidence indicate that IGF-I, IGF-II and their receptor IGFR1 are important mediators of the malignant phenotype. Plasma levels of IGF-I have been found to be a predictor of prostate cancer risk and similar epidemiological studies strongly link plasma IGF-I levels with breast, colon and lung cancer risk. Overexpression of Insulin-like Growth Factor Receptor-1 has also been demonstrated in several cancer cell lines and tumor tissues. IGFR1 is overexpressed in breast, cervical, colorectal and lung cancer cell lines.

Therapeutic anti-cancer antibodies may be combined with a further chemotherapeutic agent which, for example, may provide significant therapeutic benefits to the patient, for example, by targeting a metabolic pathway distinct from that targeted by the antibody.

### SUMMARY OF THE INVENTION

The present invention provides, in part, a composition comprising (e.g., a kit optionally further including a package insert with instructions for use) an isolated antibody or antigen-binding fragment thereof (e.g., antibody such as a humanized antibody) comprising CDR-L1; CDR-L2; and CDR-L3 in a light chain immunoglobulin variable region comprising the amino acid sequence set forth in SEQ ID NO: 1; and/or, CDR-H1; CDR-H2; and CDR-H3 in a heavy chain immunoglobulin variable region comprising the amino acid sequence set forth in SEQ ID NO: 2; in association with cisplatin, pemetrexed, gemcitabine and/or irinotecan (e.g., pemetrexed and cisplatin); e.g., wherein the isolated antibody or antigen-binding fragment thereof (e.g., antibody such as a humanized antibody; e.g., dalotuzumab) comprises a light chain immunoglobulin variable region comprising: CDR-L1 comprising the amino acid sequence set forth in SEQ ID NO: 3; CDR-L2 comprising the amino acid sequence set forth in SEQ ID NO: 4; and CDR-L3 comprising the amino acid sequence set forth in SEQ ID NO: 5; and/or a heavy chain immunoglobulin variable region comprising: CDR-H1 comprising the amino acid sequence set forth in SEQ ID NO: 6; CDR-H2 comprising the amino acid sequence set forth in SEQ ID NO: 7; and CDR-H3 comprising the amino acid sequence set forth in SEQ ID NO: 8; e.g., wherein the antibody or fragment comprises a light chain immunoglobulin variable region comprising the amino acid sequence set forth in SEQ ID NO: 1; and/or a heavy chain immunoglobulin variable region comprising the amino acid sequence set forth in SEQ ID NO: 2. In an embodiment of the invention, the composition comprises or excludes a further chemotherapeutic agent such as, for example, a HER2 antagonist, aprepitant, topotecan, 131-I-TM-601, 13-cis-retinoic acid, 4-hydroxytamoxifen, 5-deooxyuridine, 5′-deoxy-5-fluorouridine, 5-fluorouracil and leucovorin; PEG-labeled irinotecan, 6-mercaptopurine, 7-hydroxy staurosporine, a CDK inhibitor, a combination of irinotecan, 5-fluorouracil and leucovorin, a farnesyl protein transferase inhibitor, a lutenizing hormone-releasing hormone agonist, a MEK inhibitor, a progestational agent, a progestin, a Raf inhibitor, a selective estrogen receptor modulator, a VEGFR inhibitor, anti-VEGFR-2 antibody, abraxane, zotarolimus, ABX-EGF antibody, acolbifene, ADS-100380, axitinib, alprazolam, ALT-110, altretamine, amifostine, aminoglutethimide, amrubicin, amsacrine, an anti-EGFR antibody, an antiestrogen, an anti-HER2 antibody, an aromatase inhibitor, an EGF Receptor antagonist, an interferon, an antiemetic, an mTOR inhibitor, an NK-1 receptor antagonist, anagrelide, anastrazole, anastrozole, angiostatin, ARQ-197, arzoxifene, Asparaginase, AT-9263, atrasentan, ficlatuzumab, barasertib, selumetinib, cediranib, Bacillus Calmette-Guerin (BCG) vaccine, batabulin, BAY-43-9006, BC-210, bevacizumab, Bio 111, BIO 140, bleomycin, BMS-214662, ixabepilone, BMS-275291, BMS-310705, bortezimib, buserelin, busulfan, calcitriol, camptothecin, canertinib, capecitabine, carboplatin, carmustine, casopitant, CC 8490, cetuximab, CG-1521, CG-781, dovitinib, chlamydocin, chlorambucil, cilengitide, cimitidine, cladribine, clodronate, COL-3, conjugated estrogens, CP-724714, cyclophosphamide, cyproterone, cytarabine, cytosine arabinoside, dacarbazine, dactinomycin, darbepoetin alfa, dasatanib, daunorubicin, decatanib, denileukin, deoxycoformycin, DES (diethylstilbestrol), dexamethasone, diethylstilbestrol, diftitox, diphenhydramine, DN-101, docetaxel, dolasetron, doxorubicin, droloxifene, dronabinol, droperidol, edotecarin, edotreotide, pelitinib, cilengitide, endostatin, enzastaurin, epirubicin, epithilone B, epoetin alfa, ERA-923, erbitux, erlotinib, erythropoietin, estradiol, estramustine, etoposide, everolimus, exemestane, finasteride, depsipeptide, flavopiridol, floxuridine, fludarabine, fludrocortisones, fluorouracil, fluoxymesterone, flutamide, FOLFOX regimen, folinic acid, fulvestrant, gefitinib, gimatecan, goserelin acetate, gossypol, granisetron, GSK461364, GSK690693, lapatinib, haloperidol, neratinib, HMR-3339, hydroxyprogesterone caproate, hydroxyurea, hydroxyzine, idarubicin, idoxifene, ifosfamide, L-glutamine L-tryptophan dipeptide, imatinib, anti-KDR antibody IMC-1C11, INO 1001, interleukin-12, IPdR, ipilimumab, JNJ-16241199, KRN951, KRX-0402, L-779450, lapatanib, lasofoxifene, Lep-etu, letrozole, leucovorin, leuprolide, leuprolide acetate, levamisole, lomustine, lonafarnib, lorazepam, lucanthone, LY292223, LY292696, LY293646, LY293684, LY294002, marimastat, mechlorethamine, medroxyprogesterone acetate, megestrol acetate, melphalan, mercaptopurine, mesna, methotrexate, methylprednisolone, metoclopramide, mithramycin, mitomycin, mitotane, mitoxantrone, tozasertib, MLN8054, neovastat, netupitant, neuradiab, nilotinib, nilutamide, nolatrexed, dacinostat, oblimersen, octreotide, ofatumumab, ondansetron, oregovomab, oxaliplatin, paclitaxel, palonosetron, pamidronate, panitumumab, pazopanib, PD184352, PD0325901, pegfilgrastim, pentostatin, danusertib, phenylalanine mustard, pipendoxifene, PKI-166, plicamycin, porfimer, procarbazine, prochlorperazine, PTK787/ZK 222584, R-763, raloxifene, raltitrexed, rapamycin, razoxin, rituximab, romidepsin, berubicin, rubitecan, SB-556629, L-alanosine, Seliciclib, sirolimus, sorafenib, spironolactone, squalamine, streptozocin, semaxinib, SU6668, suberoyl analide hydroxamic acid, sunitinib, sunitinib malate, mubritinib, talampanel, tamoxifen, temozolomide, temsirolimus, teniposide, tesmilifene, testosterone, tetrandrine, thalidomide, thioguanine, thiotepa, ticilimumab, tipifarnib, canfosfamide, topotecan, toremifene citrate, trabectedin, trastuzumab, tretinoin, trichostatin A, triptorelin pamoate, tropisetron, bazedoxifene, uracil mustard, valproic acid, valrubicin, vandetanib, vatalanib, VEGF trap, vinblastine, vincristine, vindesine, vinorelbine, vitaxin, vitespan, vorinostat, VX-745, wortmannin, diphtheria toxin (S525F variant) conjugated to transferrin, zanolimumab, ZK186619, ZK-304709, ZM336372,

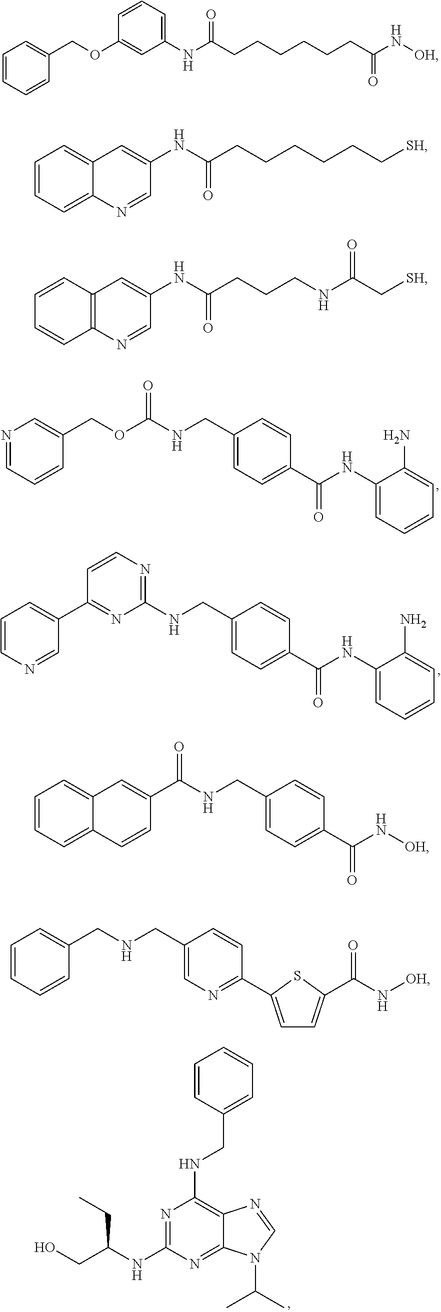

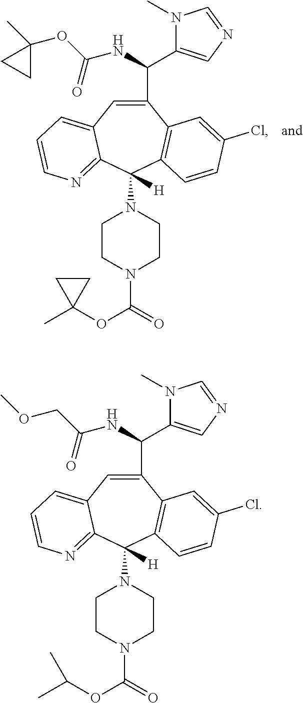 [SMILES: Cn1cncc1[C@H](NC(=O)OC1(C)CC1)C1=Cc2cccnc2[C@@H](N2CCN(C(=O)OC3(C)CC3)CC2)c2ccc(Cl)cc21 ; "and" ; COCC(=O)N[C@H](C1=Cc2cccnc2[C@@H](N2CCN(C(=O)OC(C)C)CC2)c2ccc(Cl)cc21)c1cncn1C]

The present invention also provides a method for treating or preventing a cancer (e.g., a malignant tumor) whose growth, survival and/or metastasis is mediated by IGF1R expression and/or activity (e.g., ovarian cancer, pancreatic cancer, breast cancer, prostate cancer, osteosarcoma, rhabdomyosarcoma, neuroblastoma, multiple myeloma, lung cancer, colorectal cancer and cervical cancer) in a subject in need of such treating or preventing (e.g., a human) comprising administering, to the subject, a therapeutically effective amount of a composition comprising an isolated antibody or antigen-binding fragment thereof (e.g., an antibody such as a humanized antibody; e.g., dalotuzumab) comprising CDR-L1; CDR-L2; and CDR-L3 in a light chain immunoglobulin variable region comprising the amino acid sequence set forth in SEQ ID NO: 1; and/or, CDR-H1; CDR-H2; and CDR-H3 in a heavy chain immunoglobulin variable region comprising the amino acid sequence set forth in SEQ ID NO: 2; in association with cisplatin, pemetrexed, gemcitabine and/or irinotecan (e.g., pemetrexed and cisplatin) whereby the cancer growth, survival and/or metastasis is inhibited or prevented; e.g., wherein the isolated antibody or antigen-binding fragment thereof comprises: CDR-L1 comprising the amino acid sequence set forth in SEQ ID NO: 3; CDR-L2 comprising the amino acid sequence set forth in SEQ ID NO: 4; and CDR-L3 comprising the amino acid sequence set forth in SEQ ID NO: 5; and/or a heavy chain immunoglobulin variable region comprising: CDR-H1 comprising the amino acid sequence set forth in SEQ ID NO: 6; CDR-H2 comprising the amino acid sequence set forth in SEQ ID NO: 7; and CDR-H3 comprising the amino acid sequence set forth in SEQ ID NO: 8; e.g., wherein the antibody or fragment comprises a light chain immunoglobulin variable region comprising the amino acid sequence set forth in SEQ ID NO: 1; and/or a heavy chain immunoglobulin variable region comprising the amino acid sequence set forth in SEQ ID NO: 2. In an embodiment of the invention, the subject is administered the composition of the present invention in association with a further chemotherapeutic agent such as a HER2 antagonist, aprepitant, topotecan, 131-I-TM-601, 13-cis-retinoic acid, 4-hydroxytamoxifen, 5-deooxyuridine, 5′-deoxy-5-fluorouridine, 5-fluorouracil and leucovorin; PEG-labeled irinotecan, 6-mercaptopurine, 7-hydroxy staurosporine, a CDK inhibitor, a combination of irinotecan, 5-fluorouracil and leucovorin, a farnesyl protein transferase inhibitor, a lutenizing hormone-releasing hormone agonist, a MEK inhibitor, a progestational agent, a progestin, a Raf inhibitor, a selective estrogen receptor modulator, a VEGFR inhibitor, anti-VEGFR-2 antibody, abraxane, zotarolimus, ABX-EGF antibody, acolbifene, ADS-100380, axitinib, alprazolam, ALT-110, altretamine, amifostine, aminoglutethimide, amrubicin, amsacrine, an anti-EGFR antibody, an antiestrogen, an anti-HER2 antibody, an aromatase inhibitor, an EGF Receptor antagonist, an interferon, an antiemetic, an mTOR inhibitor, an NK-1 receptor antagonist, anagrelide, anastrazole, anastrozole, angiostatin, ARQ-197, arzoxifene, Asparaginase, AT-9263, atrasentan, ficlatuzumab, barasertib, selumetinib, cediranib, Bacillus Calmette-Guerin (BCG) vaccine, batabulin, BAY-43-9006, BC-210, bevacizumab, Bio 111, BIO 140, bleomycin, BMS-214662, ixabepilone, BMS-275291, BMS-310705, bortezimib, buserelin, busulfan, calcitriol, camptothecin, canertinib, capecitabine, carboplatin, carmustine, casopitant, CC 8490, cetuximab, CG-1521, CG-781, dovitinib, chlamydocin, chlorambucil, cilengitide, cimitidine, cladribine, clodronate, COL-3, conjugated estrogens, CP-724714, cyclophosphamide, cyproterone, cytarabine, cytosine arabinoside, dacarbazine, dactinomycin, darbepoetin alfa, dasatanib, daunorubicin, decatanib, denileukin, deoxycoformycin, DES (diethylstilbestrol), dexamethasone, diethylstilbestrol, diftitox, diphenhydramine, DN-101, docetaxel, dolasetron, doxorubicin, droloxifene, dronabinol, droperidol, edotecarin, edotreotide, pelitinib, cilengitide, endostatin, enzastaurin, epirubicin, epithilone B, epoetin alfa, ERA-923, erbitux, erlotinib, erythropoietin, estradiol, estramustine, etoposide, everolimus, exemestane, finasteride, depsipeptide, flavopiridol, floxuridine, fludarabine, fludrocortisones, fluorouracil, fluoxymesterone, flutamide, FOLFOX regimen, folinic acid, fulvestrant, gefitinib, gimatecan, goserelin acetate, gossypol, granisetron, GSK461364, GSK690693, lapatinib, haloperidol, neratinib, HMR-3339, hydroxyprogesterone caproate, hydroxyurea, hydroxyzine, idarubicin, idoxifene, ifosfamide, L-glutamine L-tryptophan dipeptide, imatinib, anti-KDR antibody IMC-1C11, INO 1001, interleukin-12, IPdR, ipilimumab, JNJ-16241199, KRN951, KRX-0402, L-779450, lapatanib, lasofoxifene, Lep-etu, letrozole, leucovorin, leuprolide, leuprolide acetate, levamisole, lomustine, lonafarnib, lorazepam, lucanthone, LY292223, LY292696, LY293646, LY293684, LY294002, marimastat, mechlorethamine, medroxyprogesterone acetate, megestrol acetate, melphalan, mercaptopurine, mesna, methotrexate, methylprednisolone, metoclopramide, mithramycin, mitomycin, mitotane, mitoxantrone, tozasertib, MLN8054, neovastat, netupitant, neuradiab, nilotinib, nilutamide, nolatrexed, dacinostat, oblimersen, octreotide, ofatumumab, ondansetron, oregovomab, oxaliplatin, paclitaxel, palonosetron, pamidronate, panitumumab, pazopanib, PD184352, PD0325901, pegfilgrastim, pentostatin, danusertib, phenylalanine mustard, pipendoxifene, PKI-166, plicamycin, porfimer, procarbazine, prochlorperazine, PTK787/ZK 222584, R-763, raloxifene, raltitrexed, rapamycin, razoxin, rituximab, romidepsin, berubicin, rubitecan, SB-556629, L-alanosine, Seliciclib, sirolimus, sorafenib, spironolactone, squalamine, streptozocin, semaxinib, SU6668, suberoyl analide hydroxamic acid, sunitinib, sunitinib malate, mubritinib, talampanel, tamoxifen, temozolomide, temsirolimus, teniposide, tesmilifene, testosterone, tetrandrine, thalidomide, thioguanine, thiotepa, ticilimumab, tipifarnib, canfosfamide, topotecan, toremifene citrate, trabectedin, trastuzumab, tretinoin, trichostatin A, triptorelin pamoate, tropisetron, bazedoxifene, uracil mustard, valproic acid, valrubicin, vandetanib, vatalanib, VEGF trap, vinblastine, vincristine, vindesine, vinorelbine, vitaxin, vitespan, vorinostat, VX-745, wortmannin, diphtheria toxin (S525F variant) conjugated to transferrin, zanolimumab, ZK186619, ZK-304709, ZM336372,

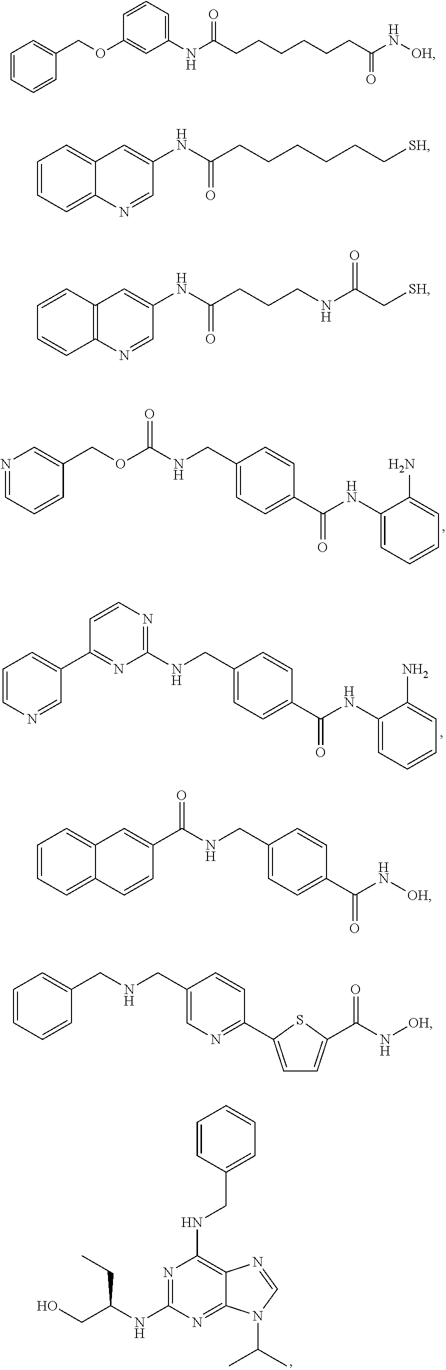

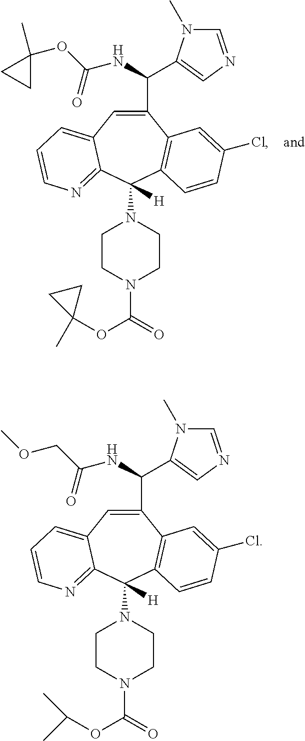 [SMILES: Cn1cncc1[C@H](NC(=O)OC1(C)CC1)C1=Cc2cccnc2[C@@H](N2CCN(C(=O)OC3(C)CC3)CC2)c2ccc(Cl)cc21 ; "and" ; COCC(=O)N[C@H](C1=Cc2cccnc2[C@@H](N2CCN(C(=O)OC(C)C)CC2)c2ccc(Cl)cc21)c1cncn1C]

The present invention further provides a method for making a composition comprising an isolated antibody or antigen-binding fragment thereof comprising CDR-L1; CDR-L2; and CDR-L3 in a light chain immunoglobulin variable region comprising the amino acid sequence set forth in SEQ ID NO: 1; and/or, CDR-H1; CDR-H2; and CDR-H3 in a heavy chain immunoglobulin variable region comprising the amino acid sequence set forth in SEQ ID NO: 2 (wherein the antibody or fragment is the product of a process comprising transforming a host cell with an expression vector having polynucleotides encoding the light and heavy immunoglobulin chains of the antibody or antigen-binding fragment operably linked to one or more promoters that drive expression of the chains; and culturing the transformed host cell in a medium under conditions that allow expression of the chains; and, optionally, isolating the chains of the antibody or fragment from the host cell and/or the medium); in association with cisplatin, pemetrexed, gemcitabine and/or irinotecan; comprising placing the antibody or antigen-binding fragment thereof in association with the pemetrexed, cisplatin, gemcitabine and/or irinotecan. In an embodiment of the invention, CDR-L1 comprises the amino acid sequence set forth in SEQ ID NO: 3; CDR-L2 comprises the amino acid sequence set forth in SEQ ID NO: 4; and CDR-L3 comprises the amino acid sequence set forth in SEQ ID NO: 5; and/or a heavy chain immunoglobulin variable region comprises: CDR-H1 comprises the amino acid sequence set forth in SEQ ID NO: 6; CDR-H2 comprises the amino acid sequence set forth in SEQ ID NO: 7; and CDR-H3 comprises the amino acid sequence set forth in SEQ ID NO: 8; e.g., wherein the antibody or fragment comprises a light chain immunoglobulin variable region comprising the amino acid sequence set forth in SEQ ID NO: 1; and/or a heavy chain immunoglobulin variable region comprises the amino acid sequence set forth in SEQ ID NO: 2.

### BRIEF DESCRIPTION OF THE FIGURES

FIG. 1. Kaplan-Meier plot of progression free survival (PFS) of pancreatic cancer patients treated with gemcitabine+dalotuzumab (A); gemcitabine+erlotinib+dalotuzumab (B); or gemcitabine+erlotinib (C; control).

FIG. 2. Kaplan-Meier plot of overall survival (OS) of pancreatic cancer patients treated with gemcitabine+dalotuzumab (A); gemcitabine+erlotinib+dalotuzumab (B); or gemcitabine+erlotinib (C; control).

### DETAILED DESCRIPTION OF THE INVENTION

The present invention provides methods for treating or preventing cancers whose growth, survival and/or metastasis is mediated by IGF1R expression and/or activity by administering a composition of the present invention that comprises an antibody or antigen-binding fragment thereof that specifically binds to IGF1R (e.g., dalotuzumab) in association with cisplatin, pemetrexed, gemcitabine and/or irinotecan. In an embodiment of the invention, dalotuzumab is in association with gemcitabine or with pemetrexed and cisplatin. Compositions comprising the antibody or fragment in association with cisplatin, pemetrexed, gemcitabine and/or irinotecan are also part of the present invention.

### Molecular biology

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

A polypeptide or protein comprises two or more amino acids.

The term “isolated protein”, “isolated polypeptide” is a protein or polypeptide that by virtue of its origin or source of derivation (1) is not associated with naturally associated components that accompany it in its native state, (2) is free of other proteins from the same species, (3) is expressed by a cell from a different species, (4) was isolated or purified e.g., by a technician and/or (5) does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

A “polynucleotide”, “nucleic acid” or “nucleic acid molecule” includes double-stranded and single-stranded DNA and RNA.

A “polynucleotide sequence”, “nucleic acid sequence” or “nucleotide sequence” is a series of nucleotide bases (also called “nucleotides”) in a nucleic acid, such as DNA or RNA, and means any chain of two or more nucleotides.

An amino acid sequence comprises two or more amino acids.

A “coding sequence” or a sequence “encoding” an expression product, such as an RNA or polypeptide, is a nucleotide sequence that, when expressed, results in production of the product.

The term “host cell” includes any cell of any organism that is selected, modified, transfected, transformed, grown, or used or manipulated in any way, for the production of a substance by the cell, for example the expression or replication, by the cell, of a gene, a DNA or RNA sequence, a protein or an enzyme. A host cell can be a eukaryotic cell or prokaryotic cell. A eukaryotic cell can be, for example, a Chinese hamster ovary cell (e.g., CHO-K1 or DXB11), a HeLa cell, an NIH 3T3 cell, or a yeast or fungal cell, such as S. cerevisiae, any Pichia cell, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia, Saccharomyces cerevisiae, Saccharomyces, Hansβnula polymorpha, Kluyveromyces, Kluyveromyces lactis, Candida albicans, Aspergillus nidulans, Aspergillus niger, Aspergillus oryzae, Trichoderma reesei, Chrysosporium lucknowense, Fusarium, Fusarium gramineum, Fusarium venenatum or Neuraspora crassa. A prokaryotic cell can be, for example, a bacterial cell such as E. coli (e.g., BL21 or BL21 DE3); see U.S. Pat. Nos. 4,952,496, 5,693,489 and 5,869,320 and in Davanloo, P., et al., (1984) Proc. Natl. Acad. Sci. USA 81, 2035-2039; Studier, F. W., et al., (1986) J. Mol. Biol. 189: 113-130; Rosenberg, A. H., et al., (1987) Gene 56: 125-135; and Dunn, J. J., et al., (1988) Gene 68: 259 which are herein incorporated by reference.

The present invention includes methods and compositions comprising anti-IGF1R antibodies and antigen-binding fragments thereof. The term “anti-IGF1R antibody” or the like refers to a full antibody that binds specifically to IGF1R (e.g., human IGF1R); for example, monoclonal antibodies, polyclonal antibodies, bispecific antibodies, chimeric antibodies, recombinant antibodies, anti-idiotypic antibodies, humanized antibodies and bispecific antibodies. The term “antigen-binding fragment” of an antibody encompasses a fragment of an antibody, typically including at least a portion of the antigen-binding or variable regions (e.g., one or more CDRs) of the parental antibody, that retains at least some of the binding specificity of the parental antibody. Examples of antigen-binding fragments of an antibody include, but are not limited to, Fab, Fab′, F(ab′)₂, and Fv fragments; dsFv; (dsFv)₂, ds diabodies; dsFv-dsFv′; single-chain antibody molecules, e.g., sc-Fv, sc-Fv dimers (bivalent diabodies); and bispecific diabodies.

Antibodies and antigen-binding fragments thereof bind specifically to IGF1R if they exhibit a K_{D} of about 10⁻⁸ M or a lower number (e.g., 10⁻⁹ M, 10⁻¹⁰ M, 10⁻¹¹ M, 10⁻¹²M).

### Further Chemotherapeutic Agents

A “composition of the present invention” comprises an anti-IGF1R antibody or antigen-binding fragment thereof (e.g., dalotuzumab) in association with any one or more of pemetrexed, cisplatin, gemcitabine and irinotecan; e.g., pemetrexed and cisplatin and further in association with a further chemotherapeutic agent.

In an embodiment of the invention, a composition of the present invention is in further association with any one or more of the following: erlotinib, dasatanib, nilotinib, decatanib, panitumumab, amrubicin, oregovomab, Lep-etu, nolatrexed, cediranib, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, INO 1001, IPdR, KRX-0402, lucanthone, LY 317615, neuradiab, vitespan, berubicin, L-alanosine, talampanel, atrasentan, diphtheria toxin (S525F variant) conjugated to transferrin, everolimus, trabectedin, abraxane, canfosfamide, ficlatuzumab, DN-101, pazopanib, GSK690693, berubicin, ON 0910.Na, selumetinib, GSK461364, barasertib, enzastaurin, vandetanib, ARQ-197, tozasertib, MLN8054, danusertib, R-763 or AT-9263.

In an embodiment of the invention, a composition of the present invention is in association with abraxane. Abraxane is an injectable suspension of paclitaxel protein-bound particles comprising an albumin-bound form of paclitaxel with a mean particle size of approximately 130 nanometers. Abraxane is supplied as a white to yellow, sterile, lyophilized powder for reconstitution with 20 mL of 0.9% Sodium Chloride Injection, USP prior to intravenous infusion. Each single-use vial contains 100 mg of paclitaxel and approximately 900 mg of human albumin. Each milliliter (mL) of reconstituted suspension contains 5 mg paclitaxel. Abraxane is free of solvents and is free of cremophor (polyoxyethylated castor oil).

In an embodiment of the invention, a composition of the present invention is in association with romidepsin (depsipeptide, FK-228), ADS-100380,

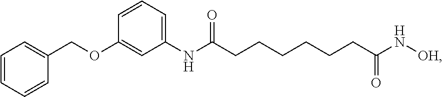 [SMILES: O=C(CCCCCCC(=O)Nc1cccc(OCc2ccccc2)c1)NO]

### CG-781, CG-1521

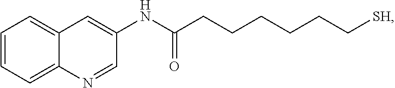 [SMILES: O=C(CCCCCCS)Nc1cnc2ccccc2c1]

SB-556629, chlamydocin, JNJ-16241199,

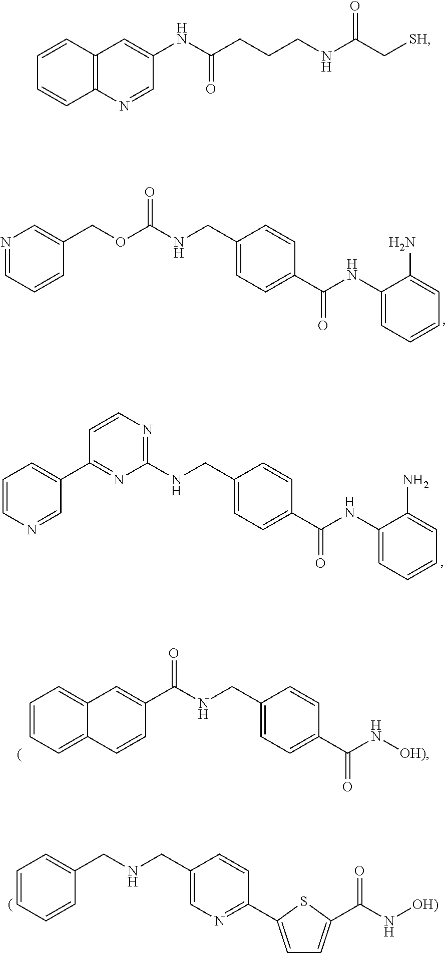

or vorinostat (SAHA).

In an embodiment of the invention, a composition of the present invention is in association with a gamma secretase inhibitor such as:

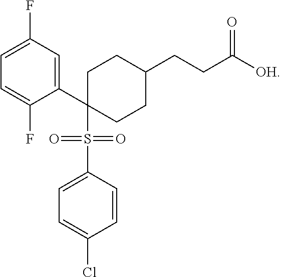 [SMILES: O=C(O)CCC1CCC(c2cc(F)ccc2F)(S(=O)(=O)c2ccc(Cl)cc2)CC1]

In an embodiment of the invention, a composition of the present invention is in association with an AKT inhibitor such as

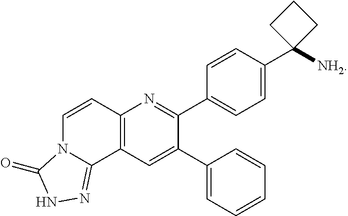

In an embodiment of the invention, a composition of the present invention is in association with an mTOR inhibitor such as

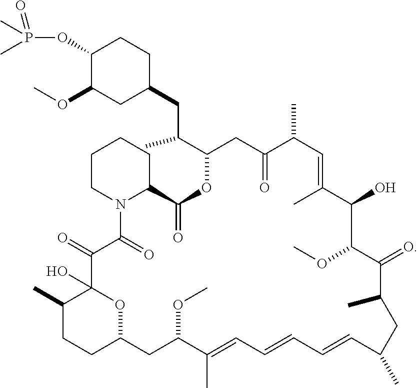

In an embodiment of the invention, a composition of the present invention is in association with an ERK inhibitor such as

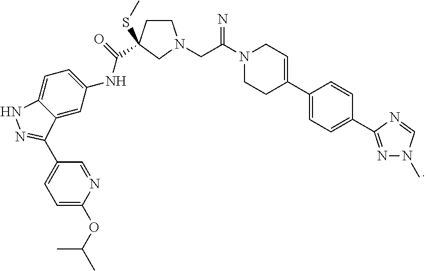 [SMILES: CS[C@@]1(C(=O)Nc2ccc3[nH]nc(-c4ccc(OC(C)C)nc4)c3c2)CCN(CC(=N)N2CC=C(c3ccc(-c4ncn(C)n4)cc3)CC2)C1]

In an embodiment of the invention, a composition of the present invention is in association with etoposide (VP-16).

In an embodiment of the invention, a composition of the present invention is in association with doxorubicin including Caelyx or Doxil® (doxorubicin HCl liposome injection; Ortho Biotech Products L.P; Raritan, N.J.). Doxil® comprises doxorubicin in STEALTH® liposome carriers which are composed of N-(carbonyl-methoxypolyethylene glycol 2000)-1,2-distearoyl-sn-glycero-3-phosphoethanolamine sodium salt (MPEG-DSPE); fully hydrogenated soy phosphatidylcholine (HSPC), and cholesterol.

In an embodiment of the invention, a composition of the present invention is in association with 5′-deoxy-5-fluorouridine.

In an embodiment of the invention, a composition of the present invention is in association with vincristine.

In an embodiment of the invention, a composition of the present invention is in association with temozolomide any CDK inhibitor such as ZK-304709, seliciclib (R-roscovitine; any MEK inhibitor such as PD0325901, selumetinib; capecitabine (5′-deoxy-5-fluoro-N-[(pentyloxy)carbonyl]-cytidine); or L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate

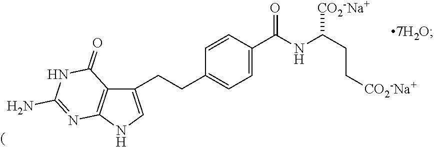

Pemetrexed disodium heptahydrate).

In an embodiment of the invention, a composition of the present invention is in association with camptothecin; Stork et al., J. Am. Chem. Soc. 93(16): 4074-4075 (1971); Beisler et al., J. Med. Chem. 14(11): 1116-1117 (1962)), or a combination of irinotecan, 5-fluorouracil and leucovorin; or PEG-labeled irinotecan.

In an embodiment of the invention, a composition of the present invention is in association with the FOLFOX regimen components (oxaliplatin, together with infusional fluorouracil and folinic acid (Chaouche et al., Am. J. Clin. Oncol. 23(3):288-289 (2000); de Gramont et al., J. Clin. Oncol. 18(16):2938-2947 (2000)).

In an embodiment of the invention, a composition of the present invention is in association with an antiestrogen such as tamoxifen; sold as Nolvadex® by AstraZeneca Pharmaceuticals LP; Wilmington, Del. or toremifene citrate; sold as Fareston® by Shire US, Inc.; Florence, Ky.

In an embodiment of the invention, a composition of the present invention is in association with an aromatase inhibitor such as anastrazole; sold as Arimidex® by AstraZeneca Pharmaceuticals LP; Wilmington, Del., exemestane; sold as Aromasin® by Pharmacia Corporation; Kalamazoo, Mich. or letrozole; sold as Femara® by Novartis Pharmaceuticals Corporation; East Hanover, N.J.

In an embodiment of the invention, a composition of the present invention is in association with an estrogen such as DES (diethylstilbestrol), estradiol; sold as Estrol® by Warner Chilcott, Inc.; Rockaway, N.J. or conjugated estrogens (sold as Premarin® by Wyeth Pharmaceuticals Inc.; Philadelphia, Pa.).

In an embodiment of the invention, a composition of the present invention is in association with one or more anti-angiogenesis agents including bevacizumab (Avastin™; Genentech; San Francisco, Calif.), the anti-KDR antibody IMC-1C11, other VEGFR inhibitors such as: dovitinib, vatalanib (PTK787; ZK-222584), axitinib; and the VEGF trap (AVE-0005), a soluble decoy receptor comprising portions of VEGF receptors 1 and 2.

In an embodiment of the invention, a composition of the present invention is in association with a LHRH (Lutenizing hormone-releasing hormone) agonist such as the acetate salt of [D-Ser(Bu t)₆, Azgly 10] (pyro-Glu-His-Trp-Ser-Tyr-D-Ser(Bu t)-Leu-Arg-Pro-Azgly-NH₂ acetate [C₅₉H₈₄O₁₄.(C₂H₄O₂)ₓ where x=1 to 2.4]; goserelin acetate; sold as Zoladex® by AstraZeneca UK Limited; Macclesfield, England, leuprolide acetate; sold as Eligard® by Sanofi-Synthelabo Inc.; New York, N.Y.) or triptorelin pamoate; sold as Trelstar® by Pharmacia Company, Kalamazoo, Mich.

In an embodiment of the invention, a composition of the present invention is in association with sunitinib or sunitinib malate.

In an embodiment of the invention, a composition of the present invention is in association with a progestational agent such as medroxyprogesterone acetate; sold as Provera® by Pharmacia & Upjohn Co.; Kalamazoo, Mich., hydroxyprogesterone caproate, megestrol acetate or progestins.

In an embodiment of the invention, a composition of the present invention is in association with a selective estrogen receptor modulator (SERM) such as raloxifene; sold as Evista® by Eli Lilly and Company; Indianapolis, Ind.

In an embodiment of the invention, a composition of the present invention is in association with an anti-androgen including, but not limited to: bicalutamide; sold at CASODEX® by AstraZeneca Pharmaceuticals LP; Wilmington, Del.; flutamide; 2-methyl-N-[4-nitro-3 (trifluoromethyl)phenyl]propanamide; sold as Eulexin® by Schering Corporation; Kenilworth, N.J.; nilutamide; sold as Nilandron® by Aventis Pharmaceuticals Inc.; Kansas City, Mo. and Megestrol)

In an embodiment of the invention, a composition of the present invention is in association with one or more inhibitors which antagonize the action of the EGF Receptor or HER2, including, but not limited to, CP-724714

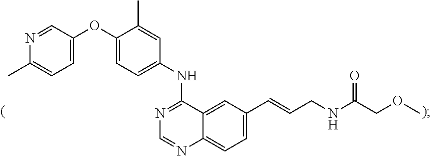

TAK-165, mubritinib; neratinib; erlotinib, Hidalgo et al., J. Clin. Oncol. 19(13): 3267-3279 (2001)), Lapatanib (GW2016; Rusnak et al., Molecular Cancer Therapeutics 1:85-94 (2001); N-{3-Chloro-4-[(3-fluorobenzyl)oxy]phenyl}-6-[5-({[2-(methylsulfonyl)ethyl]amino}methyl)-2-furyl]-4-quinazolinamine; PCT Application No. WO99/35146), Canertinib (Erlichman et al., Cancer Res. 61(2):739-48 (2001); Smaill et al., J. Med. Chem. 43(7):1380-97 (2000)), ABX-EGF antibody (Abgenix, Inc.; Freemont, Calif.; Yang et al., Cancer Res. 59(6):1236-43 (1999); Yang et al., Crit. Rev Oncol Hematol. 38(1):17-23 (2001)), erbitux (U.S. Pat. No. 6,217,866; IMC-C225, cetuximab; Imclone; New York, N.Y.), pelitinib (Wissner et al., J. Med. Chem. 46(1): 49-63 (2003)), PKI-166

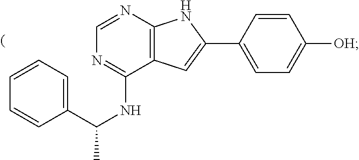 [SMILES: C[C@@H](Nc1ncnc2[nH]c(-c3ccc(O)cc3)cc12)c1ccccc1]

3GP-75166), lapatinib, any anti-EGFR antibody and any anti-HER2 antibody.

In an embodiment of the invention, a composition of the present invention is in association with lonafarnib.

In an embodiment of the invention, a composition of the present invention is in association with one or more FPT inhibitors such as:

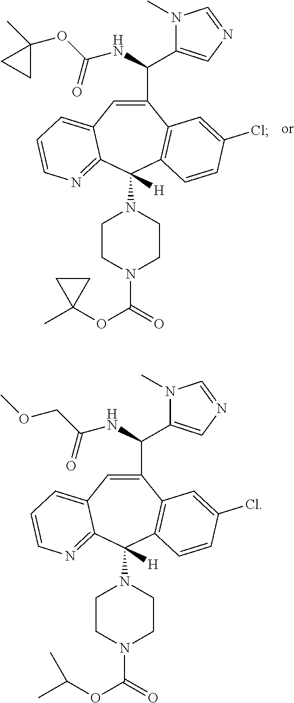 [SMILES: Cn1cncc1[C@H](NC(=O)OC1(C)CC1)C1=Cc2cccnc2[C@@H](N2CCN(C(=O)OC3(C)CC3)CC2)c2ccc(Cl)cc21 ; "or" ; COCC(=O)N[C@H](C1=Cc2cccnc2[C@@H](N2CCN(C(=O)OC(C)C)CC2)c2ccc(Cl)cc21)c1cncn1C]

Other FPT inhibitors include BMS-214662

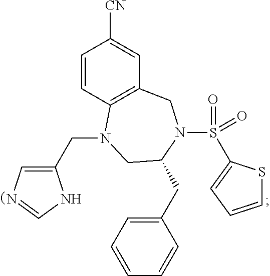 [SMILES: N#Cc1ccc2c(c1)CN(S(=O)(=O)c1cccs1)[C@H](Cc1ccccc1)CN2Cc1cnc[nH]1]

Hunt et al., J. Med. Chem. 43(20):3587-95 (2000); Dancey et al., Curr. Pharm. Des. 8:2259-2267 (2002); (R)-7-cyano-2,3,4,5-tetrahydro-1-(1H-imidazol-4-ylmethyl)-3-(phenylmethyl)-4-(2-thienylsulfonyl)-1H-1,4-benzodiazepine)) and R155777 (tipifarnib; Garner et al., Drug Metab. Dispos. 30(7):823-30 (2002); Dancey et al., Curr. Pharm. Des. 8:2259-2267 (2002); (B)-6-[amino(4-chlorophenyl)(1-methyl-1H-imidazol-5-yl)-methyl]-4-(3-chlorophenyl)-1-methyl-2(1H)-quinolinone]; sold as Zarnestra™; Johnson & Johnson; New Brunswick, N.J.).

In an embodiment of the invention, a composition of the present invention is in association with Amifostine; dacinostat; Atadja et al., Cancer Research 64: 689-695 (2004)), suberoyl analide hydroxamic acid, Valproic acid; Michaelis et al., Mol. Pharmacol. 65:520-527 (2004)), trichostatin A, FK-228 (Furumai et al., Cancer Research 62: 4916-4921 (2002)), sunitinib (Mendel et al., Clin. Cancer Res. 9(1):327-37 (2003)), sorafenib, KRN951, Aminoglutethimide; Amsacrine; Anagrelide; Anastrozole; Asparaginase; Bacillus Calmette-Guerin (BCG) vaccine (Gamido et al., Cytobios. 90(360):47-65 (1997)); bleomycin; Buserelin; (Busulfan; 1,4-butanediol, dimethanesulfonate; sold as Busulfex® by ESP Pharma, Inc.; Edison, N.J.); Carboplatin; sold as Paraplatin® by Bristol-Myers Squibb; Princeton, N.J.; Carmustine; Chlorambucil; Cladribine; Clodronate; Cyclophosphamide; Cyproterone; Cytarabine; Dacarbazine; Dactinomycin; Daunorubicin; Diethylstilbestrol; Epirubicin; Fludarabine; Fludrocortisone; Fluoxymesterone; Flutamide; Hydroxyurea; Idarubicin; Ifosfamide; Imatinib; sold as Gleevec® by Novartis Pharmaceuticals Corporation; East Hanover, N.J.; Leucovorin; Leuprolide; Levamisole; Lomustine; Mechlorethamine; Melphalan; Mercaptopurine; Mesna; Methotrexate; Mitomycin; Mitotane; Mitoxantrone; Nilutamide; octreotide; edotreotide (yttrium-90 labeled or unlabeled); oxaliplatin; Pamidronate; Pentostatin; plicamycin; porfimer; Procarbazine; Raltitrexed; Rituximab; Streptozocin; Teniposide; Testosterone; Thalidomide; Thioguanine; Thiotepa; Tretinoin; Vindesine or 13-cis-retinoic acid.

In an embodiment of the invention, a composition of the present invention is in association with one or more of any of: phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, semaxinib, SU6668, cilengitide, interleukin-12, L-glutamine L-tryptophan dipeptide, angiostatin, vitaxin, droloxifene, idoxyfene, spironolactone, finasteride, cimitidine, trastuzumab, denileukin, diftitox, gefitinib, bortezimib, paclitaxel, docetaxel, epithilone B, ixabepilone (see e.g., Lee et al., Clin. Cancer Res. 7:1429-1437 (2001)), BMS-310705, droloxifene (3-hydroxytamoxifen), 4-hydroxytamoxifen, pipendoxifene, ERA-923, arzoxifene, fulvestrant, acolbifene, lasofoxifene (CP-336156), idoxifene, bazedoxifene, HMR-3339, ZK186619, topotecan, PTK787/ZK 222584 (Thomas et al., Semin Oncol. 30(3 Suppl 6):32-8 (2003)), the humanized anti-VEGF antibody Bevacizumab, VX-745 (Haddad, Curr Opin. Investig. Drugs 2(8):1070-6 (2001)), PD 184352 (Sebolt-Leopold, et al. Nature Med. 5: 810-816 (1999)), any mTOR inhibitor, rapamycin, sirolimus, 40-O-(2-hydroxyethyl)-rapamycin, temsirolimus (Sehgal et al., Med. Res. Rev., 14:1-22 (1994); Elit, Curr. Opin. Investig. Drugs 3(8):1249-53 (2002)), zotarolimus; BC-210, LY294002, LY292223, LY292696, LY293684, LY293646 (Vlahos et al., J. Biol. Chem. 269(7): 5241-5248 (1994)), wortmannin, ZM336372, L-779,450, any Raf inhibitor disclosed in Lowinger et al., Curr. Pharm Des. 8:2269-2278 (2002); flavopiridol (Senderowicz, Oncogene 19(56): 6600-6606 (2000)) or 7-hydroxy staurosporine (Senderowicz, Oncogene 19(56): 6600-6606 (2000)).

In an embodiment of the invention, a composition of the present invention is in association with one or more of any of: pegylated or unpegylated interferon alfa-2a, pegylated or unpegylated interferon alfa-2b, pegylated or unpegylated interferon alfa-2c, pegylated or unpegylated interferon alfa n-1, pegylated or unpegylated interferon alfa n-3 and pegylated, unpegylated consensus interferon or albumin-interferon-alpha.

The scope of the present invention also includes methods wherein a composition of the present invention in association with one or more antiemetics including, but not limited to, casopitant (GlaxoSmithKline), Netupitant (MGI-Helsinn) and other NK-1 receptor antagonists, palonosetron (sold as Aloxi by MGI Pharma), aprepitant (sold as Emend by Merck and Co.; Rahway, N.J.), diphenhydramine (sold as Benadryl® by Pfizer; New York, N.Y.), hydroxyzine (sold as Atarax® by Pfizer; New York, N.Y.), metoclopramide (sold as Reglan® by AH Robins Co,; Richmond, Va.), lorazepam (sold as Ativan® by Wyeth; Madison, N.J.), alprazolam (sold as Xanax® by Pfizer; New York, N.Y.), haloperidol (sold as Haldol® by Ortho-McNeil; Raritan, N.J.), droperidol (Inapsine®), dronabinol (sold as Marinol® by Solvay Pharmaceuticals, Inc.; Marietta, Ga.), dexamethasone (sold as Decadron® by Merck and Co.; Rahway, N.J.), methylprednisolone (sold as Medrol® by Pfizer; New York, N.Y.), prochlorperazine (sold as Compazine® by Glaxosmithkline; Research Triangle Park, N.C.), granisetron (sold as Kytril® by Hoffmann-La Roche Inc.; Nutley, N.J.), ondansetron (sold as Zofran® by by Glaxosmithkline; Research Triangle Park, N.C.), dolasetron (sold as Anzemet® by Sanofi-Aventis; New York, N.Y.) or tropisetron (sold as Navoban® by Novartis; East Hanover, N.J.).

Other side effects of cancer treatment include red and white blood cell deficiency. Accordingly, the present invention includes compositions of the present invention in association with an agent which treats or prevents such a deficiency, such as, e.g., pegfilgrastim, erythropoietin, epoetin alfa or darbepoetin alfa.

Other side effects of cancer treatment include diarrhea; accordingly, the present invention includes compositions of the present invention in association with an agent which treats or prevents diarrhea such as an electrolyte solution, a bulking agents such as methylcellulose, guar gum or plant fibre (e.g., bran, sterculia, isabgol, an absorbents such as methylcellulose, an anti-inflammatory drug such as bismuth subsalicylate or an opioid such as loperamide.

The present invention further comprises a method for treating or preventing any stage or type of any medical condition set forth herein by administering a composition of the present invention in association with a therapeutic procedure such as surgical tumorectomy or anti-cancer radiation treatment.

The scope of the present invention encompasses embodiments wherein compositions of the present invention comprise an anti-IGF1R antibody or antigen-binding fragment thereof and one or more of cisplatin, pemetrexed, gemcitabine and/or irinotecan as well as methods of treatment using such compositions, as discussed herein, wherein the compositions exclude any further chemotherapeutic agent or therapeutic procedure (other non-therapeutic agents may be optionally included). In an embodiment of the invention, the excluded further chemotherapeutic agent or further therapeutic procedure is any one or more of those set forth herein (e.g., erlotinib).

The term “in association with” indicates that the components of a composition of the present invention, optionally in association with a further chemotherapeutic agent, can be formulated into a single composition for simultaneous delivery or formulated separately into two or more compositions (e.g., a kit). Furthermore, each component of a composition of the present invention, optionally in association with a further chemotherapeutic agent, can be administered, in a method of the present invention, to a subject at a different time than when the other component is administered; for example, each administration may be given non-simultaneously (e.g., separately or sequentially) at several intervals over a given period of time. Moreover, separate components may be administered to a subject by the same or by a different route (e.g., orally and subcutaneously).

### Anti-IGF1R

The compositions of the present invention provides compositions comprising antibodies and antigen-binding fragmetns thereof that bind specifically with IGF1R (e.g., dalotuzumab (MK0646)) in association with cisplatin, pemetrexed, gemcitabine and/or irinotecan (e.g., pemetrexed and cisplatin). In an embodiment of the invention, the antibodies and antigen-binding fragments thereof that binds specifically to IGF1R comprise:

| LIGHT CHAIN (1) (CDRs underscored) | |
|---|---|
| (SEQ ID NO: 1) | |
| 1 | DIVMTQSPLS LPVTPGEPAS ISCRSSQSIV HSNGNTYLQW YLQKPGQSPQ |
| 51 | LLIYKVSNRL YGVPDRFSGS GSGTDFTLKI SRVEAEDVGV YYCFQGSHVP |
| 101 | WTFGQGTKVE IKRTVAAPSV FIFPPSDEQL KSGTASVVCL LNNFYPREAK |
| 151 | VQWKVDNALQ SGNSQESVTE QDSKDSTYSL SSTLTLSKAD YEKHKVYACE |
| 201 | VTHQGLSSPV TKSFNRGEC; |
| and | |
| HEAVY CHAIN (2) (CDRs underscored) | |
| (SEQ ID NO: 2) | |
| 1 | QVQLQESGPG LVKPSETLSL TCTVSGYSIT GGYLWNWIRQ PPGKGLEWIG |
| 51 | YISYDGTNNY KPSLKDRVTI SRDTSKNQFS LKLSSVTAAD TAVYYCARYG |
| 101 | RVFFDYWGQG TLVTVSSAST KGPSVFPLAP SSKSTSGGTA ALGCLVKDYF |
| 151 | PEPVTVSWNS GALTSGVHTF PAVLQSSGLY SLSSVVTVPS SSLGTQTYIC |
| 201 | NVNHKPSNTK VDKRVEPKSC DKTHTCPPCP APELLGGPSV FLFPPKPKDT |
| 251 | LMISRTPEVT CVVVDVSHED PEVKFNWYVD GVEVHNAKTK PREEQYNSTY |
| 301 | RVVSVLTVLH QDWLNGKEYK CKVSNKALPA PIEKTISKAK GQPREPQVYT |
| 351 | LPPSREEMTK NQVSLTCLVK GFYPSDIAVE WESNGQPENN YKTTPPVLDS |
| 401 | DGSFFLYSKL TVDKSRWQQG NVFSCSVMHE ALHNHYTQKS LSLSPGK; |
| and | |
| HEAVY CHAIN (3) | |
| (SEQ ID NO: 2) | |
| 1 | QVQLQESGPG LVKPSETLSL TCTVSGYSIT GGYLWNWIRQ PPGKGLEWIG |
| 51 | YISYDGTNNY KPSLKDRVTI SRDTSKNQFS LKLSSVTAAD TAVYYCARYG |
| 101 | RVFFDYWGQG TLVTVSSAST KGPSVFPLAP SSKSTSGGTA ALGCLVKDYF |
| 151 | PEPVTVSWNS GALTSGVHTF PAVLQSSGLY SLSSVVTVPS SSLGTQTYIC |
| 201 | NVNHKPSNTK VDKRVEPKSC DKTHTCPPCP APELLGGPSV FLFPPKPKDT |
| 251 | LMISRTPEVT CVVVDVSHED PEVKFNWYVD GVEVHNAKTK PREEQYNSTY |
| 301 | RVVSVLTVLH QDWLNGKEYK CKVSNKALPA PIEKTISKAK GQPREPQVYT |
| 351 | LPPSREEMTK NQVSLTCLVK GFYPSDIAVE WESNGQPENN YKTTPPVLDS |
| 401 | DGSFFLYSKL TVDKSRWQQG NVFSCSVMHE ALHNHYTQKS LSLSPGK; |
| and | |
| LIGHT CHAIN (4) | |
| (SEQ ID NO: 1) | |
| 1 | DIVMTQSPLS LPVTPGEPAS ISCRSSQSIV HSNGNTYLQW YLQKPGQSPQ |
| 51 | LLIYKVSNRL YGVPDRFSGS GSGTDFTLKI SRVEAEDVGV YYCFQGSHVP |
| 101 | WTFGQGTKVE IKRTVAAPSV FIFPPSDEQL KSGTASVVCL LNNFYPREAK |
| 151 | VQWKVDNALQ SGNSQESVTE QDSKDSTYSL SSTLTLSKAD YEKHKVYACE |
| 201 | VTHQGLSSPV TKSFNRGEC |

or an antibody or antigen binding fragment thereof comprising CDR-L1, CDR-L2, CDR-L3, CDR-H1, CDR-H2, and CDR-H3 of such chains;
e.g., wherein
CDR-L1 comprises the amino acid sequence set forth in SEQ ID NO: 3 (RSSQSIVHSNGNTYLQ)
CDR-L2 comprises the amino acid sequence set forth in SEQ ID NO: 4 (KVSNRLY)
CDR-L3 comprises the amino acid sequence set forth in SEQ ID NO: 5 (FQGSHVPWT)
CDR-H1 comprises the amino acid sequence set forth in SEQ ID NO: 6 (GGYLWN)
CDR-H2 comprises the amino acid sequence set forth in SEQ ID NO: 7 (YISYDGTNNYKPSLKD)
CDR-H3 comprises the amino acid sequence set forth in SEQ ID NO: 8 (YGRVFFDY);
and/or the light chain and heavy chain variable regions of such chains; e.g., wherein the chains are disulfide bonded as follows:

### Disulfide Bridges (Chain Number:Amino Acid Number)

1:23 to 1:93
1:139 to 1:199
1:219 to 2:220
2:22 to 2:96
2:144 to 2:200
2:261 to 2:321
2:367 to 2:425
2:226 to 3:226
2:229 to 3:229
3:22 to 3:96
3:144 to 3:200
3:261 to 3:321
3:367 to 3:425
3:220 to 4:219
4:23 to 4:93
4:139 to 4:199.

See U.S. Pat. No. 7,241,444.

In an embodiment of the invention, the antibodies and antigen-binding fragments thereof that binds specifically to IGF1R comprise any of the light chains and/or heavy chains set forth below (CDRs solid underscored; signal peptides dash underscored).

19D12/15H12 Light Chain
(SEQ ID NO: 9)

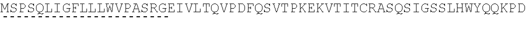

QSPKLLIKYASQSLSGVPSRFSGSGSGTDFTLTINSLEAEDAAAYYCHQSSRLPHTFGGG
TKVEIKRT
19D12/15H12 Heavy Chain
(SEQ ID NO: 10)

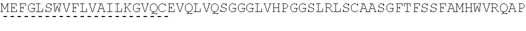

GKGLEWISVIDTRGATYYADSVKGRFTISRDNAKNSLYLQMNSLRAEDMAVYYCARLGNF
YYGMDVWGQGTTVTVSS
19D12/15H12 Light Chain-C (LCC)
(SEQ ID NO: 11)

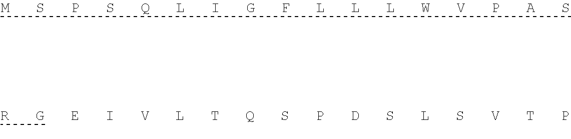

G E R V T I T C R A S Q S I G S S
L H W Y Q Q K P G Q S P K L L I K
Y A S Q S L S G V P S R F S G S G
S G T D F T L T I S S L E A E D A
A A Y Y C H Q S S R L P H T F G Q
G T K V E I K R T
19D12/15H12 Light Chain-D (LCD)
(SEQ ID NO: 12)

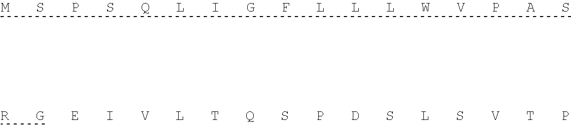

G E R V T I T C R A S Q S I G S S
L H W Y Q Q K P G Q S P K L L I K
Y A S Q S L S G V P S R F S G S G
S G T D F T L T I S S L E A E D F
A V Y Y C H Q S S R L P H T F G Q
G T K V E I K R T
19D12/15H12 Light Chain-E (LCE)
(SEQ ID NO: 13)

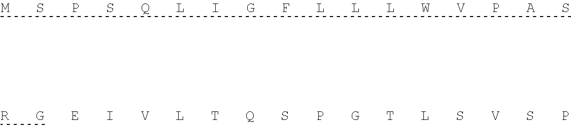

G E R A T L S C R A S Q S I G S S
L H W Y Q Q K P G Q A P R L L I K
Y A S Q S L S G I P D R F S G S G
S G T D F T L T I S R L E P E D A
A A Y Y C H Q S S R L P H T F G Q
G T K V E I K R T
19D12/15H12 Light Chain-F (LCF)
(SEQ ID NO: 14)

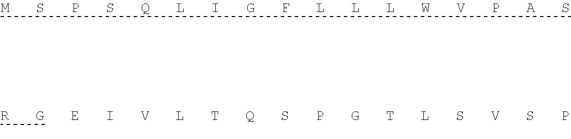

G E R A T L S C R A S Q S I G S S
L H W Y Q Q K P G Q A P R L L I K
Y A S Q S L S G I P D R F S G S G
S G T D F T L T I S R L E P E D F
A V Y Y C H Q S S R L P H T F G Q
G T K V E I K R T
19D12/15H12 heavy chain-A (HCA)
(SEQ ID NO: 15)

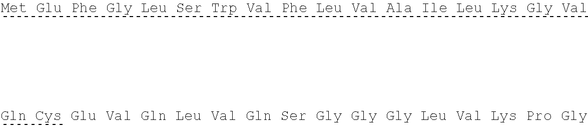

Gly Ser Leu Arg Leu Ser Cys Ala Ala Ser Gly Phe Thr Phe Ser Ser Phe
Ala Met His Trp Val Arg Gln Ala Pro Gly Lys Gly Leu Glu Trp Ile Ser
Val Ile Asp Thr Arg Gly Ala Thr Tyr Tyr Ala Asp Ser Val Lys Gly Arg
Phe Thr Ile Ser Arg Asp Asn Ala Lys Asn Ser Leu Tyr Leu Gln Met Asn
Ser Leu Arg Ala Glu Asp Thr Ala Val Tyr Tyr Cys Ala Arg Leu Gly Asn
Phe Tyr Tyr Gly Met Asp Val Trp Gly Gln Gly Thr Thr Val Thr Val Ser
Ser
19D12/15H12 heavy chain-B (HCB)
(SEQ ID NO: 16)

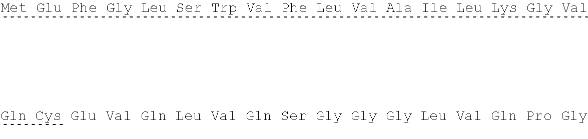

Gly Ser Leu Arg Leu Ser Cys Ala Ala Ser Gly Phe Thr Phe Ser Ser Phe
Ala Met His Trp Val Arg Gln Ala Pro Gly Lys Gly Leu Glu Trp Ile Ser
Val Ile Asp Thr Arg Gly Ala Thr Tyr Tyr Ala Asp Ser Val Lys Gly Arg
Phe Thr Ile Ser Arg Asp Asn Ala Lys Asn Ser Leu Tyr Leu Gln Met Asn
Ser Leu Arg Ala Glu Asp Thr Ala Val Tyr Tyr Cys Ala Arg Leu Gly Asn
Phe Tyr Tyr Gly Met Asp Val Trp Gly Gln Gly Thr Thr Val Thr Val Ser
Ser

(see international application publication no. WO2003/100008 which is incorporated herein by reference in its entirety) or an antibody or antigen binding fragment thereof comprising CDR-L1, CDR-L2, CDR-L3, CDR-H1, CDR-H2, and CDR-H3 of such chains and/or the light chain and heavy chain variable regions of such chains.

### Therapeutic Methods, Formulations, Dosage and Administration

The present invention provides methods for treating or preventing an IGF1R-mediated medical condition using a composition comprising anti-IGF1R and any one or more of pemetrexed, cisplatin, gemcitabine and irinotecan. In an embodiment of the invention, the subject suffers from a cancer or malignancy, e.g., that expresses IGF1R and/or whose survival, growth and/or metastasis is mediated by IGF1R activity and/or expression; such as ovarian cancer, pancreatic cancer, breast cancer (e.g., estrogen receptor positive or negative breast cancer), prostate cancer, osteosarcoma, rhabdomyosarcoma, neuroblastoma, multiple myeloma, lung cancer (e.g., non-small cell lung cancer, small cell lung cancer, adenosquamous cell lung cancer, squamous cell lung cancer, recurrent non-small cell lung cancer, stage IIIB non-small cell lung cancer, stage IV non-small cell lung cancer), colorectal cancer (e.g., advanced, metastatic or mucinous adenocarcinoma) or cervical cancer.

The term “subject” or “patient” refers to a mammal such as a human (e.g., a human adult or child) or a mouse, rat, rabbit, dog or other canine, horse, goat or primate such as a monkey, chimpanzee or gorilla.

The IGF1R inhibitors discussed herein (e.g., anti-IGF1R antibodies and antigen-binding fragments thereof) and compositions thereof are, in an embodiment of the invention, administered at a therapeutically effective dosage. The term “therapeutically effective amount” or “therapeutically effective dosage” means that amount or dosage of an agent that will elicit a biological or medical response of a tissue, system, patient or subject that is being sought by the administrator (such as a researcher, doctor or veterinarian) which includes any measurable alleviation of the signs, symptoms and/or clinical indicia of a medical disorder, such as cancer (e.g., tumor growth, survival and/or metastasis) including the prevention, slowing or halting of progression of the medical disorder to any degree. For example, in one embodiment of the invention, a “therapeutically effective dosage” of any anti-IGF1R antibody or antigen-binding fragment thereof discussed herein (e.g., dalotuzumab) is between about 0.3 and 20 mg/kg of body weight (e.g., 0.3 mg/kg, 1 mg/kg, 3 mg/kg, 5 mg/kg, 10 mg/kg, 15 mg/kg, 20 mg/kg), e.g., about once per week to about once every 3 weeks (e.g., about once every 1 week or once every 2 weeks or once every 3 weeks). In an embodiment of the invention, dalotuzumab is administered once per week at a rate of 10 mg/kg body weight. The therapeutically effective dosage of an anti-IGF1R antibody or antigen-binding fragment thereof or any further therapeutic agent is, when possible, as set forth in Physicians Desk Reference 2010; Thomson Reuters; 64 edition (Nov. 15, 2009); and/or in Physicians' Desk Reference 2009; Thomson Reuters; 63rd edition (Nov. 30, 2008) or in the prescribing information of the relevant drug label (if available), such as the US FDA drug label.

In an embodiment of the invention, gemcitabine is administered (e.g., intravenously) at about 1000 mg/m² e.g., over 30 minutes on days 1 and 8 of each 21-day cycle; or about 1250 mg/m², e.g., over 30 minutes on days 1 and 8 of each 21-day cycle; or about 1000 mg/m² e.g., over 30 minutes on days 1, 8, and 15 of each 28-day cycle or in a 3-week schedule at about 1250 mg/m² e.g., over 30 minutes on days 1 and 8 of each 21-day cycle; or about 1000 mg/m², e.g., over 30 minutes once weekly for up to 7 weeks e.g., followed by a week of rest from treatment. In an embodiment of the invention, subsequent cycles include infusions once weekly for 3 consecutive weeks out of every 4 weeks.

In an embodiment of the invention, pemetrexed is administered (e.g., intravenously) at about 500 mg/m², e.g., on day 1 of each 21-day cycle, e.g., in combination with cisplatin 75 mg/m², e.g., intravenously, e.g., beginning 30 minutes after pemetrexed administration; or at about 500 mg/m², e.g., on day 1 of each 21-day cycle; e.g., wherein dose reductions or discontinuation are done based on toxicities from the preceding cycle of therapy.

In an embodiment of the invention, erlotinib is administered at a dose of about 100 mg.

In an embodiment of the present invention, patients are monitored during treatment with a composition of the invention for neutropenia, dermatitis acneiform, leucopenia, nausea, decreased appetite, diarrhoea, stomatitis, fatigue, paronychia, constipation, lymphopenia, weight decrease, hyperglycaemia and/or hypoalbuminaemia. If an adverse event is observed, a treating physician can them determine whether to alter or discontinue the treatment regimen.

The present invention also includes methods for making a composition of the present invention which comprises placing the anti-IGF1R antibody or antigen-binding fragment thereof in association with the pemetrexed, cisplatin, gemcitabine and/or irinotecan, e.g., in a kit. In an embodiment of the invention, the antibody or fragment is the product of a process wherein a host cell (e.g., a Chinese hamster ovary cell or a Pichia cell such as Pichia pastoris) is transformed with one or more expression vectors having one or more polynucleotides encoding the light and/or heavy immunoglobulin chains of the antibody or fragment operably linked to one or more promoters that drive expression of the chains; the transformed host cell is cultured in a medium under conditions that allow expression of the chains, and, optionally, the chains of the antibody or fragment are isolated from the host cell and/or the medium.

The present invention includes methods for using a pharmaceutical composition comprising an anti-IGF1R antibody or antigen-binding fragment thereof. The pharmaceutical compositions may be prepared by any methods well known in the art of pharmacy; see, e.g., Gilman, et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Easton, Pa.; Avis, et al., (eds.) (1993) Pharmaceutical Dosage Forms: Parenteral Medications Dekker, New York; Lieberman, et al., (eds.) (1990) Pharmaceutical Dosage Forms: Tablets Dekker, New York; and Lieberman, et al., (eds.) (1990), Pharmaceutical Dosage Forms: Disperse Systems Dekker, New York.

A pharmaceutical composition, e.g., containing an anti-IGF1R antibody or antigen-binding fragment thereof can be prepared using conventional pharmaceutically acceptable excipients and additives and conventional techniques. Such pharmaceutically acceptable excipients and additives include non-toxic compatible fillers, binders, disintegrants, buffers, preservatives, anti-oxidants, lubricants, flavorings, thickeners, coloring agents, emulsifiers and the like. All routes of administration are contemplated including, but not limited to, parenteral (e.g., subcutaneous, intratumoral, intravenous, intraperitoneal, intramuscular) and non-parenteral (e.g., oral, transdermal, intranasal, intraocular, sublingual, inhalation, rectal and topical).

Injectables can be prepared in conventional forms, either as liquid solutions or suspensions, solid forms suitable for solution or suspension in liquid prior to injection, or as emulsions. The injectables, solutions and emulsions can also contain one or more excipients. Excipients are, for example, water, sugar, buffer, salt (e.g., NaCl), amino acids (e.g., histidine or glycine), saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

In an embodiment of the invention, pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN-80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

In an embodiment of the invention, preparations for parenteral administration can include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

### EXAMPLES

The following information is provided for more clearly describing the present invention and should not be construed to limit the present invention. Any and all of the compositions and methods described below fall within the scope of the present invention.

### Example 1

### Clinical Testing of Dalotuzumab/Gemcitabine Combination in Human Cancer Patients

Human volunteers suffering from pancreatic cancer were separated into three arms and dosed as set forth below.

Arm A: gemcitabine (1000 mg/m² Days 1, 8, 15)+dalotuzumab (10 mg/kg; once a week);
Arm B: gemcitabine (1000 mg/m² Days 1, 8, 15)+erlotinib (100 mg Days 1-28)+dalotuzumab (10 mg/kg; once a week);
Arm C: gemcitabine (1000 mg/m² Days 1, 8, 15)+erlotinib (100 mg Days 1-28) (Control)

The median progression free survivals (PFS) and overall survivals (OS) for patients in each arm (95% confidence interval) were determined to be the following. The number of patients in each arm (n) and range of values observed (bracketed values) are also shown. All calculations were performed on Stata/IC 10.0, Survival Analysis Module (Stata Corp., College Station, Tex.).

### Median PFS

Arm A (n=15): 17 wks [7, 34]
Arm B (n=20): 8 wks [8, 18]
Arm C (n=8): 8 wks [3, 22]

### Median OS

A (n=15, median follow-up=101 wks): 48 wks [14, 82]
B (n=20, median follow-up=70 wks): 30 wks [18, 56]
C (n=8, median follow-up=42 wks): 26 wks [8, 40]
*subjects who progressed in arm C were permitted to cross-over and receive treatment with the Arm B regimen.

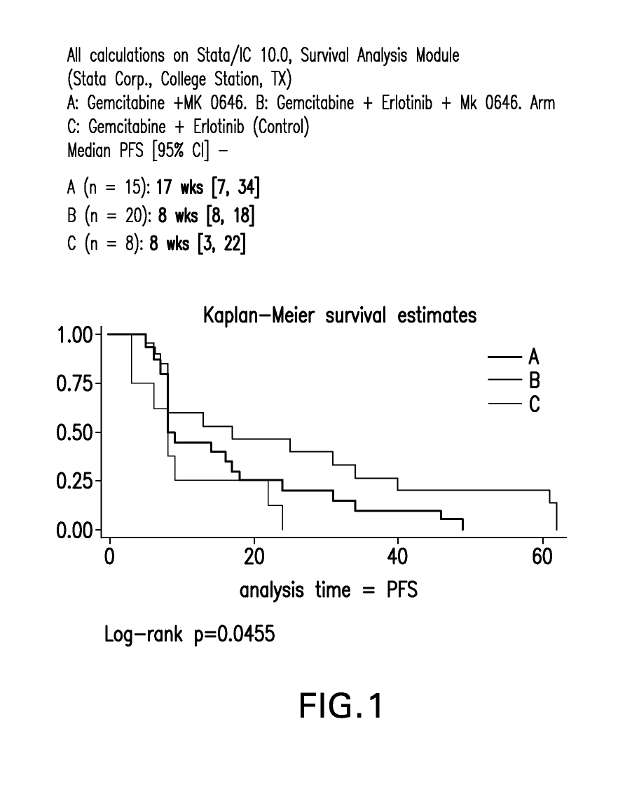

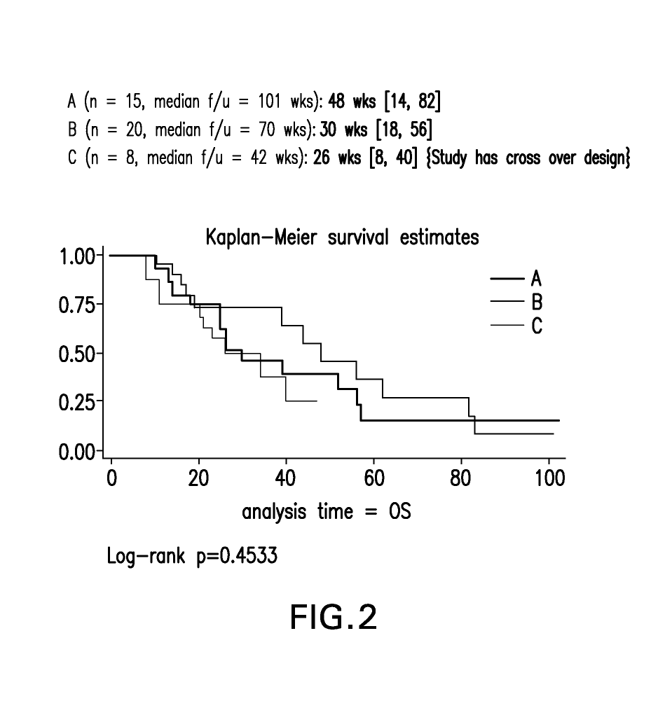

The median PFS and median OS data summarized above are also graphically represented in FIGS. 1 and 2. The data in FIG. 1 suggests that patients receiving dalotuzumab and gemcitabine benefited from a greater PFS than patients receiving dalotuzumab, gencitabine and erlotinib. This effect will be the subject of further investigation so as to determine whether it is reproduced and statistically significant.

The present invention is not to be limited in scope by the specific embodiments described herein. Indeed, the scope of the present invention includes embodiments specifically set forth herein and other embodiments not specifically set forth herein; the embodiments specifically set forth herein are not necessarily intended to be exhaustive. Various modifications of the invention in addition to those described herein will become apparent to those skilled in the art from the foregoing description. Such modifications are intended to fall within the scope of the claims.

Patents, patent applications, publications, product descriptions, and protocols are cited throughout this application, the disclosures of which are incorporated herein by reference in their entireties for all purposes.

We claim:

1. A composition comprising an isolated antibody or antigen-binding fragment thereof comprising CDR-L1; CDR-L2; and CDR-L3 in a light chain immunoglobulin variable region comprising the amino acid sequence set forth in SEQ ID NO: 1;
and/or,
CDR-H1; CDR-H2; and CDR-H3 in a heavy chain immunoglobulin variable region comprising the amino acid sequence set forth in SEQ ID NO: 2;
in association with gemcitabine and/or irinotecan.

2. The composition of claim 1 comprising dalotuzumab in association with gemcitabine and/or irinotecan.

3. The composition of claim 1 wherein the antibody or fragment is in association with gemcitabine and irinotecan.

4. The composition of claim 1 wherein said antibody or fragment is in association with a carrier.

5. The composition of claim 4 wherein the carrier comprises a buffer.

6. The composition of claim 1 wherein said antibody or fragment is in association with gemcitabine.

7. The composition of claim 1 wherein said antibody or fragment is in association with irinotecan.

8. The composition claim 1 wherein the antibody or fragment is an antibody.

9. The composition of claim 8 wherein the antibody is a humanized antibody.

10. The composition claim 1 wherein the antibody or fragment comprises a light chain immunoglobulin variable region comprising the amino acid sequence set forth in SEQ ID NO: 1; and/or a heavy chain immunoglobulin variable region comprising the amino acid sequence set forth in SEQ ID NO: 2.

11. The composition of claim 1 which further comprises a further chemotherapeutic agent.

12. The composition of claim 11 where the further chemotherapeutic agent is one or more members selected from the group consisting of: a HER2 antagonist, aprepitant, topotecan, 131-I-TM-601, 13-cis-retinoic acid, 4-hydroxytamoxifen, 5-deooxyuridine, 5′-deoxy-5-fluorouridine, 5-fluorouracil and leucovorin; PEG-labeled irinotecan, 6-mercaptopurine, 7-hydroxy staurosporine, a CDK inhibitor, a combination of irinotecan, 5-fluorouracil and leucovorin, a farnesyl protein transferase inhibitor, a lutenizing hormone-releasing hormone agonist, a MEK inhibitor, a gamma secretase inhibitor, an ERK inhibitor, an AKT inhibitor, a progestational agent, a progestin, a Raf inhibitor, a selective estrogen receptor modulator, a VEGFR inhibitor, anti-VEGFR-2 antibody, abraxane, zotarolimus, ABX-EGF antibody, acolbifene, ADS-100380, axitinib, alprazolam, ALT-110, altretamine, amifostine, aminoglutethimide, amrubicin, amsacrine, an anti-EGFR antibody, an antiestrogen, an anti-HER2 antibody, an aromatase inhibitor, an EGF Receptor antagonist, an interferon an antiemetic, an mTOR inhibitor, an NK-1 receptor antagonist, anagrelide, anastrazole, anastrozole, angiostatin, ARQ-197, arzoxifene, Asparaginase, AT-9263, atrasentan, ficlatuzumab, barasertib, selumetinib, cediranib, Bacillus Calmette-Guerin (BCG) vaccine, batabulin, BC-210, bevacizumab, Bio 111, BIO 140, bleomycin, BMS-214662, ixabepilone, BMS-275291, BMS-310705, bortezimib, buserelin, busulfan, calcitriol, camptothecin, canertinib, capecitabine, carboplatin, carmustine, casopitant, CC 8490, cetuximab, CG-1521, CG-781, dovitinib, chlamydocin, chlorambucil, cilengitide, cimitidine, cladribine, clodronate, COL-3, conjugated estrogens, CP-724714, cyclophosphamide, cyproterone, cytarabine, cytosine arabinoside, dacarbazine, dactinomycin, darbepoetin alfa, dasatanib, daunorubicin, decatanib, denileukin, deoxycoformycin, DES (diethylstilbestrol), dexamethasone, diethylstilbestrol, diftitox, diphenhydramine, DN-101, docetaxel, dolasetron, doxorubicin, droloxifene, dronabinol, droperidol, edotecarin, edotreotide, pelitinib, cilengitide, endostatin, enzastaurin, epirubicin, epithilone B, epoetin alfa, ERA-923, erbitux, erlotinib, erythropoietin, estradiol, estramustine, etoposide, everolimus, exemestane, finasteride, romidepsin, flavopiridol, floxuridine, fludarabine, fludrocortisones, fluorouracil, fluoxymesterone, flutamide, FOLFOX regimen, folinic acid, fulvestrant, gefitinib, gimatecan, goserelin acetate, gossypol, granisetron, GSK461364, GSK690693, lapatinib, haloperidol, neratinib, HMR-3339, hydroxyprogesterone caproate, hydroxyurea, hydroxyzine, idarubicin, idoxifene, ifosfamide, L-glutamine L-tryptophan dipeptide, imatinib, anti-KDR antibody IMC-1C11, INO 1001, interleukin-12, IPdR, ipilimumab, JNJ-16241199, KRN951, KRX-0402, L-779450, lapatanib, lasofoxifene, Lep-etu, letrozole, leucovorin, leuprolide, leuprolide acetate, levamisole, lomustine, lonafarnib, lorazepam, lucanthone, LY292223, LY292696, LY293646, LY293684, LY294002, marimastat, mechlorethamine, medroxyprogesterone acetate, megestrol acetate, melphalan, mercaptopurine, mesna, methotrexate, methylprednisolone, metoclopramide, mithramycin, mitomycin, mitotane, mitoxantrone, tozasertib, MLN8054, neovastat, netupitant, neuradiab, nilotinib, nilutamide, nolatrexed, dacinostat, oblimersen, octreotide, ofatumumab, ondansetron, oregovomab, oxaliplatin, paclitaxel, palonosetron, pamidronate, panitumumab, pazopanib, PD184352, PD0325901, pegfilgrastim, pentostatin, danusertib, phenylalanine mustard, pipendoxifene, PKI-166, plicamycin, porfimer, procarbazine, prochlorperazine, R-763, raloxifene, raltitrexed, rapamycin, razoxin, rituximab, berubicin, rubitecan, SB-556629, L-alanosine, Seliciclib, sirolimus, sorafenib, spironolactone, squalamine, streptozocin, semaxinib, SU6668, suberoyl analide hydroxamic acid, sunitinib, sunitinib malate, mubritinib, talampanel, tamoxifen, temozolomide, temsirolimus, teniposide, tesmilifene, testosterone, tetrandrine, thalidomide, thioguanine, thiotepa, ticilimumab, tipifarnib, canfosfamide, topotecan, toremifene citrate, trabectedin, trastuzumab, tretinoin, trichostatin A, triptorelin pamoate, tropisetron, bazedoxifene, uracil mustard, valproic acid, valrubicin, vandetanib, vatalanib, VEGF trap, vinblastine, vincristine, vindesine, vinorelbine, vitaxin, vitespan, vorinostat, VX-745, wortmannin, diphtheria toxin (S525F variant) conjugated to transferrin, zanolimumab, ZK186619, ZK-304709, ZM336372, an electrolyte solution, a bulking agent, methylcellulose, guar gum, plant fibre, an anti-inflammatory drug, bismuth subsalicylate, an opioid, loperamide,

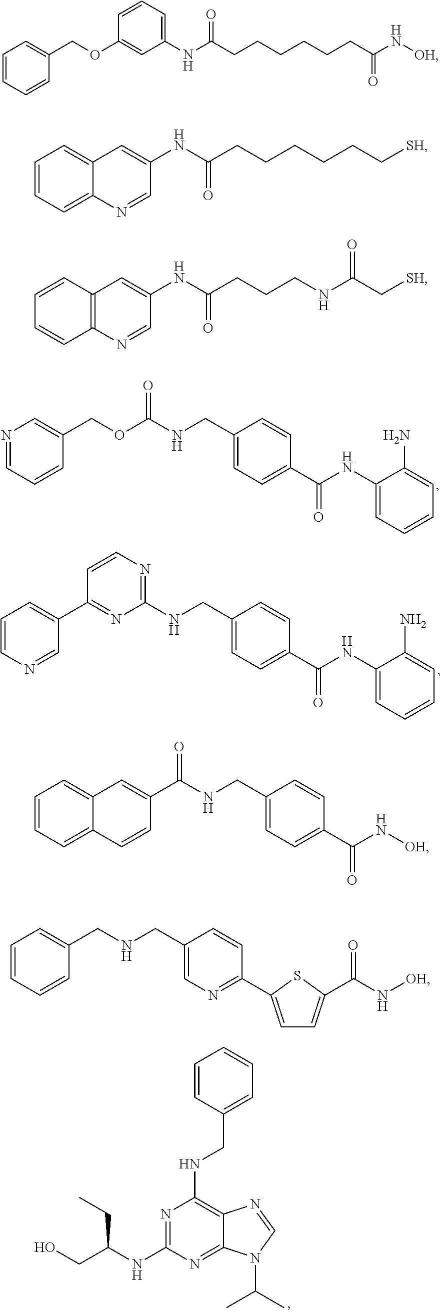

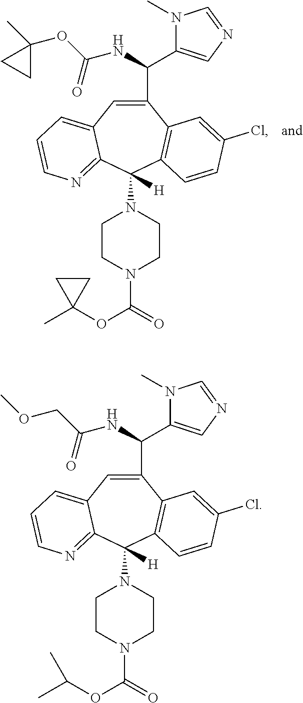 [SMILES: Cn1cncc1[C@H](NC(=O)OC1(C)CC1)C1=Cc2cccnc2[C@@H](N2CCN(C(=O)OC3(C)CC3)CC2)c2ccc(Cl)cc21 ; "and" ; COCC(=O)N[C@H](C1=Cc2cccnc2[C@@H](N2CCN(C(=O)OC(C)C)CC2)c2ccc(Cl)cc21)c1cncn1C]

13. A method for treating or preventing a cancer whose growth, survival and/or metastasis is mediated by IGF1R expression and/or activity in a subject comprising administering, to the subject, a therapeutically effective amount of a composition comprising an isolated antibody or antigen-binding fragment thereof comprising CDR-L1; CDR-L2; and CDR-L3 in a light chain immunoglobulin variable region comprising the amino acid sequence set forth in SEQ ID NO: 1;
and/or,
CDR-H1; CDR-H2; and CDR-H3 in a heavy chain immunoglobulin variable region comprising the amino acid sequence set forth in SEQ ID NO: 2;
in association with gemcitabine and/or irinotecan.

14. The method of claim 13 wherein the composition comprises dalotuzumab in association with gemcitabine and/or irinotecan.

15. The method of claim 13 wherein the antibody or fragment comprises a light chain immunoglobulin variable region comprising the amino acid sequence set forth in SEQ ID NO: 1; and/or a heavy chain immunoglobulin variable region comprising the amino acid sequence set forth in SEQ ID NO: 2.

16. The method of claim 13 wherein the cancer is a member selected from the group consisting of ovarian cancer, pancreatic cancer, breast cancer, prostate cancer, osteosarcoma, rhabdomyosarcoma, neuroblastoma, multiple myeloma, lung cancer, colorectal cancer and cervical cancer.

17. The method of claim 13 wherein the antibody or fragment is in association with gemcitabine.

18. The method of claim 13 wherein the antibody or fragment is an antibody.

19. The method of claim 18 wherein the antibody is a humanized antibody.

20. The method of claim 13 wherein the composition is administered in further association with a further chemotherapeutic agent.

21. The method of claim 20 wherein the further chemotherapeutic agent is one or more members selected from the group consisting of: a HER2 antagonist, aprepitant, topotecan, 131-I-TM-601, 13-cis-retinoic acid, 4-hydroxytamoxifen, 5-deooxyuridine, 5′-deoxy-5-fluorouridine, 5-fluorouracil and leucovorin; PEG-labeled irinotecan, 6-mercaptopurine, 7-hydroxy staurosporine, a CDK inhibitor, a combination of irinotecan, 5-fluorouracil and leucovorin, a farnesyl protein transferase inhibitor, a lutenizing hormone-releasing hormone agonist, a MEK inhibitor, a gamma secretase inhibitor, an ERK inhibitor, an AKT inhibitor, a progestational agent, a progestin, a Raf inhibitor, a selective estrogen receptor modulator, a VEGFR inhibitor, anti-VEGFR-2 antibody, abraxane, zotarolimus, ABX-EGF antibody, acolbifene, ADS-100380, axitinib, alprazolam, ALT-110, altretamine, amifostine, aminoglutethimide, amrubicin, amsacrine, an anti-EGFR antibody, an antiestrogen, an anti-HER2 antibody, an aromatase inhibitor, an EGF Receptor antagonist, an interferon an antiemetic, an mTOR inhibitor, an NK-1 receptor antagonist, anagrelide, anastrazole, anastrozole, angiostatin, ARQ-197, arzoxifene, Asparaginase, AT-9263, atrasentan, ficlatuzumab, barasertib, selumetinib, cediranib, Bacillus Calmette-Guerin (BCG) vaccine, batabulin, BC-210, bevacizumab, Bio 111, BIO 140, bleomycin, BMS-214662, ixabepilone, BMS-275291, BMS-310705, bortezimib, buserelin, busulfan, calcitriol, camptothecin, canertinib, capecitabine, carboplatin, carmustine, casopitant, CC 8490, cetuximab, CG-1521, CG-781, dovitinib, chlamydocin, chlorambucil, cilengitide, cimitidine, cladribine, clodronate, COL-3, conjugated estrogens, CP-724714, cyclophosphamide, cyproterone, cytarabine, cytosine arabinoside, dacarbazine, dactinomycin, darbepoetin alfa, dasatanib, daunorubicin, decatanib, denileukin, deoxycoformycin, DES (diethylstilbestrol), dexamethasone, diethylstilbestrol, diftitox, diphenhydramine, DN-101, docetaxel, dolasetron, doxorubicin, droloxifene, dronabinol, droperidol, edotecarin, edotreotide, pelitinib, cilengitide, endostatin, enzastaurin, epirubicin, epithilone B, epoetin alfa, ERA-923, erbitux, erlotinib, erythropoietin, estradiol, estramustine, etoposide, everolimus, exemestane, finasteride, romidepsin, flavopiridol, floxuridine, fludarabine, fludrocortisones, fluorouracil, fluoxymesterone, flutamide, FOLFOX regimen, folinic acid, fulvestrant, gefitinib, gimatecan, goserelin acetate, gossypol, granisetron, GSK461364, GSK690693, lapatinib, haloperidol, neratinib, HMR-3339, hydroxyprogesterone caproate, hydroxyurea, hydroxyzine, idarubicin, idoxifene, ifosfamide, L-glutamine L-tryptophan dipeptide, imatinib, anti-KDR antibody IMC-1C11, INO 1001, interleukin-12, IPdR, ipilimumab, JNJ-16241199, KRN951, KRX-0402, L-779450, lapatanib, lasofoxifene, Lep-etu, letrozole, leucovorin, leuprolide, leuprolide acetate, levamisole, lomustine, lonafarnib, lorazepam, lucanthone, LY292223, LY292696, LY293646, LY293684, LY294002, marimastat, mechlorethamine, medroxyprogesterone acetate, megestrol acetate, melphalan, mercaptopurine, mesna, methotrexate, methylprednisolone, metoclopramide, mithramycin, mitomycin, mitotane, mitoxantrone, tozasertib, MLN8054, neovastat, netupitant, neuradiab, nilotinib, nilutamide, nolatrexed, dacinostat, oblimersen, octreotide, ofatumumab, ondansetron, oregovomab, oxaliplatin, paclitaxel, palonosetron, pamidronate, panitumumab, pazopanib, PD184352, PD0325901, pegfilgrastim, pentostatin, danusertib, phenylalanine mustard, pipendoxifene, PKI-166, plicamycin, porfimer, procarbazine, prochlorperazine, R-763, raloxifene, raltitrexed, rapamycin, razoxin, rituximab, berubicin, rubitecan, SB-556629, L-alanosine, Seliciclib, sirolimus, sorafenib, spironolactone, squalamine, streptozocin, semaxinib, SU6668, suberoyl analide hydroxamic acid, sunitinib, sunitinib malate, mubritinib, talampanel, tamoxifen, temozolomide, temsirolimus, teniposide, tesmilifene, testosterone, tetrandrine, thalidomide, thioguanine, thiotepa, ticilimumab, tipifarnib, canfosfamide, topotecan, toremifene citrate, trabectedin, trastuzumab, tretinoin, trichostatin A, triptorelin pamoate, tropisetron, bazedoxifene, uracil mustard, valproic acid, valrubicin, vandetanib, vatalanib, VEGF trap, vinblastine, vincristine, vindesine, vinorelbine, vitaxin, vitespan, vorinostat, VX-745, wortmannin, diphtheria toxin (S525F variant) conjugated to transferrin, zanolimumab, ZK186619, ZK-304709, ZM336372, an electrolyte solution, a bulking agent, methylcellulose, guar gum, plant fibre, an anti-inflammatory drug, bismuth subsalicylate, an opioid, loperamide,

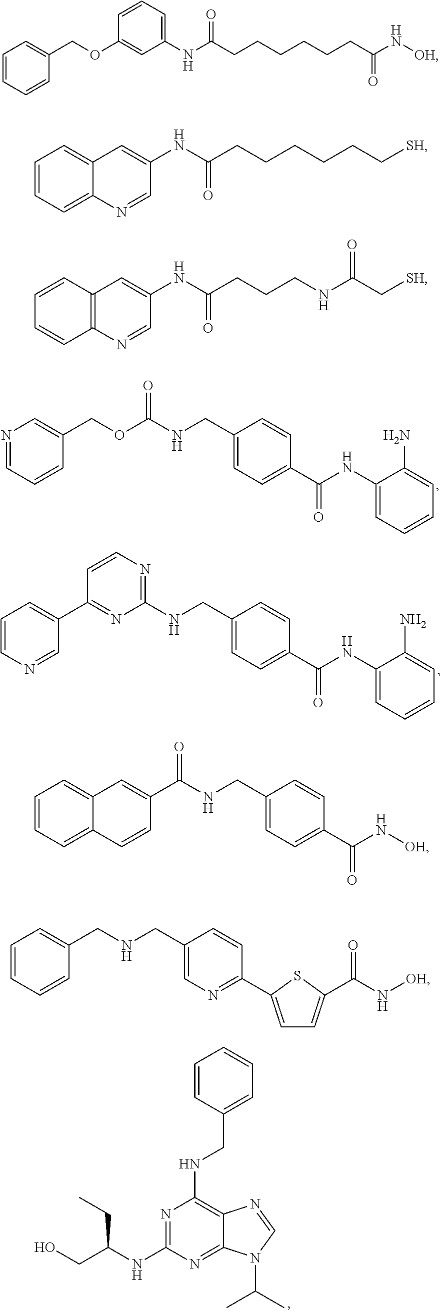

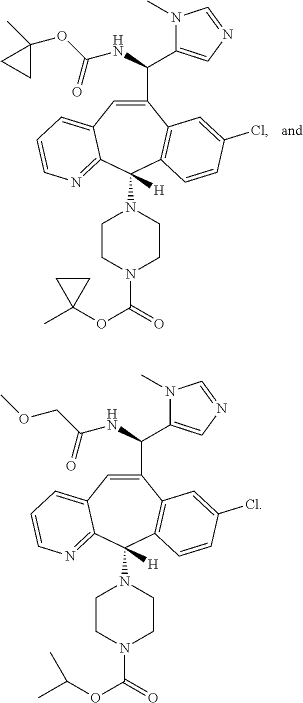 [SMILES: Cn1cncc1[C@H](NC(=O)OC1(C)CC1)C1=Cc2cccnc2[C@@H](N2CCN(C(=O)OC3(C)CC3)CC2)c2ccc(Cl)cc21 ; "and" ; COCC(=O)N[C@H](C1=Cc2cccnc2[C@@H](N2CCN(C(=O)OC(C)C)CC2)c2ccc(Cl)cc21)c1cncn1C]

22. A method for making a composition comprising an isolated antibody or antigen-binding fragment thereof comprising CDR-L1; CDR-L2; and CDR-L3 in a light chain immunoglobulin variable region comprising the amino acid sequence set forth in SEQ ID NO: 1;
and/or, CDR-H1; CDR-H2; and CDR-H3 in a heavy chain immunoglobulin variable region comprising the amino acid sequence set forth in SEQ ID NO: 2;
in association with gemcitabine and/or irinotecan; comprising placing the antibody or antigen-binding fragment thereof in association with the gemcitabine and/or irinotecan.

23. The method of claim 22 wherein the antibody or fragment is the product of a process comprising transforming a host cell with an expression vector having polynucleotides encoding the light and heavy immunoglobulin chains of the antibody or antigen-binding fragment operably linked to one or more promoters that drive expression of the chains; and culturing the transformed host cell in a medium under conditions that allow expression of the chains; and, optionally, isolating the chains of the antibody or fragment from the host cell and/or the medium.


SEQUENCE LISTING (16 sequences)

SEQ ID NO: 1 - protein, 219 aa, Artificial Sequence
  humanized immunoglobulin
DIVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 447 aa, Artificial Sequence
  humanized immunoglobulin
QVQLQESGPGLVKPSETLSLTCTVSGYSITGGYLWNWIRQPPGKGLEWIGYISYDGTNNYKPSLKDRVTISRDTSKNQFSLKLSSVTAADTAVYYCARYGRVFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 16 aa, Mus
RSSQSIVHSNGNTYLQ

SEQ ID NO: 4 - protein, 7 aa, Mus
KVSNRLY

SEQ ID NO: 5 - protein, 9 aa, Mus
FQGSHVPWT

SEQ ID NO: 6 - protein, 6 aa, Mus
GGYLWN

SEQ ID NO: 7 - protein, 16 aa, Mus
YISYDGTNNYKPSLKD

SEQ ID NO: 8 - protein, 8 aa, Mus
YGRVFFDY

SEQ ID NO: 9 - protein, 128 aa, Homo sapiens
MSPSQLIGFLLLWVPASRGEIVLTQVPDFQSVTPKEKVTITCRASQSIGSSLHWYQQKPDQSPKLLIKYASQSLSGVPSRFSGSGSGTDFTLTINSLEAEDAAAYYCHQSSRLPHTFGGGTKVEIKRT

SEQ ID NO: 10 - protein, 137 aa, Homo sapiens
MEFGLSWVFLVAILKGVQCEVQLVQSGGGLVHPGGSLRLSCAASGFTFSSFAMHWVRQAPGKGLEWISVIDTRGATYYADSVKGRFTISRDNAKNSLYLQMNSLRAEDMAVYYCARLGNFYYGMDVWGQGTTVTVSS

SEQ ID NO: 11 - protein, 128 aa, Homo sapiens
MSPSQLIGFLLLWVPASRGEIVLTQSPDSLSVTPGERVTITCRASQSIGSSLHWYQQKPGQSPKLLIKYASQSLSGVPSRFSGSGSGTDFTLTISSLEAEDAAAYYCHQSSRLPHTFGQGTKVEIKRT

SEQ ID NO: 12 - protein, 128 aa, Homo sapiens
MSPSQLIGFLLLWVPASRGEIVLTQSPDSLSVTPGERVTITCRASQSIGSSLHWYQQKPGQSPKLLIKYASQSLSGVPSRFSGSGSGTDFTLTISSLEAEDFAVYYCHQSSRLPHTFGQGTKVEIKRT

SEQ ID NO: 13 - protein, 128 aa, Homo sapiens
MSPSQLIGFLLLWVPASRGEIVLTQSPGTLSVSPGERATLSCRASQSIGSSLHWYQQKPGQAPRLLIKYASQSLSGIPDRFSGSGSGTDFTLTISRLEPEDAAAYYCHQSSRLPHTFGQGTKVEIKRT

SEQ ID NO: 14 - protein, 128 aa, Homo sapiens
MSPSQLIGFLLLWVPASRGEIVLTQSPGTLSVSPGERATLSCRASQSIGSSLHWYQQKPGQAPRLLIKYASQSLSGIPDRFSGSGSGTDFTLTISRLEPEDFAVYYCHQSSRLPHTFGQGTKVEIKRT

SEQ ID NO: 15 - protein, 137 aa, Homo sapiens
MEFGLSWVFLVAILKGVQCEVQLVQSGGGLVKPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSS

SEQ ID NO: 16 - protein, 137 aa, Homo sapiens
MEFGLSWVFLVAILKGVQCEVQLVQSGGGLVQPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSS